(19) United States
(12) Patent Application Publication
(10) Pub. No.: US 2013/0137123 A1
(43) Pub. Date: May 30, 2013

(54) USE OF HMGB1 AS A BIOLOGICAL MARKER OF BOWEL INFLAMMATORY CONDITIONS, NON-INVASIVE METHOD FOR ITS DETECTION IN FECAL SAMPLES AND KIT THEREOF

(71) Applicants: Salvatore Cucchiara, Rome (IT); Laura Stronati, Rome (IT); Roberta Vitali, Rome (IT)

(72) Inventors: Salvatore Cucchiara, Rome (IT); Laura Stronati, Rome (IT); Roberta Vitali, Rome (IT)

(73) Assignee: D.M.G. ITALIA SRL, Pomezia (RM) (IT)

(21) Appl. No.: 13/814,294

(22) PCT Filed: Aug. 1, 2011
(86) PCT No.: PCT/IT2011/000276

§ 371 Date: Feb. 5, 2013

(30) Foreign Application Priority Data
Aug. 5, 2010 (IT) RM2010A000442

(51) Int. Cl. G01N 33/68

(52) U.S. Cl. CPC G01N 33/6875
USPC 435/7.92; 435/7.9

(57) ABSTRACT

A non-invasive method for measuring a bowel inflammatory condition in humans through the presence of HMGB1 protein in fecal extracts and the involvement of such protein in the pathogenesis of chronic inflammatory bowel diseases, more specifically of Crohn's Disease (CD) and of ulcerative colitis (UC), including an analysis protocol to detect the HMGB1 presence in feces through Western blot assay or ELISA assay using an appropriate antigen-antibody. The invention also includes the colorimetric kit for implementing such a method.

The present invention relates to materials and methods for detecting and diagnosing chronic inflammatory bowel diseases (IBD “Inflammatory Bowel Disease”) in humans. In particular, it describes a non-invasive method for measuring a bowel inflammatory condition in humans through the presence of HMGB1 protein in fecal extracts and the involvement of such protein in the pathogenesis of chronic inflammatory bowel diseases, more specifically of Crohn's Disease (CD) and ulcerative colitis (UC). The invention also comprises the colorimetric kit for implementing such a method.

### FIELD OF THE INVENTION

High-mobility group box 1 (HMGB1) is a non-histone nuclear protein associated to non-histone chromatin, emerged as DAMP molecules prototype (Damage Associated Molecular Patterns) able to respond to stimuli from tissue damage by inducing an inflammatory response (1). HMGB1 is actively secreted by macrophages (2) and enterocytes (3) following pro-inflammatory stimuli such as LPS, TNFα, IL-1β, IL-6, and IL-8 (4) and it is released by necrotic cells, but not apoptotic cells (5). HMGB1, as secreted into the extracellular space, forms highly inflammatory complexes with different molecules: single-stranded DNA, LPS, IL-1β and nucleosomes, which interacting with their respective receptors, such as TLR9, TLR4, IL-1R and TLR2, activate the innate immunity. Alternatively, HMGB1 can bind, without forming complexes, the receptor for glycation end products RAGE (Receptor for Advanced Glucation End products) (6).

Extracellular HMGB1 induces the production of inflammatory mediators (4) and may play an important role in the pathogenesis of autoimmune or inflammatory diseases, including rheumatoid arthritis (7), systemic lupus erythematosus (8) and polymyositis (9). The invention described in U.S. Pat. No. 6,303,321 relates to a pharmaceutical composition for the treatment of sepsis. The pharmaceutical composition comprises as active substance an effective amount of antagonist or inhibitor of HMGB1. Preferably, among antagonists of HMGB1, antibodies binding to the HMGB1 protein, antisense sequences of the HMGB1-coding gene, and antagonists of the HMGB1 receptor are used. Thus, subject of the invention is also a method for treating sepsis comprising administering an effective amount of an antagonist of HMGB1. The invention also provides a diagnostic and prognostic method for monitoring the severity of the patient condition and predicting the likely clinical course of sepsis and related conditions for a patient with shock-like symptoms or showing associated symptoms. The diagnostic and prognostic method includes measuring the concentration of HMGB1 protein in a sample, particularly in serum or whole blood, comparing it with a standard concentration of HMGB1. Higher levels of HMGB1 are indicative of poor prognosis or likely occurrence of toxic reactions. The diagnostic method can also be applied to other tissues or fluids compartments such as cerebrospinal fluid or urine.

HMGB1 and Gastrointestinal Tract: State of the Art

Signs of stress, tissue damage or microbial antigens in the intestinal mucosa activate cells involved in the innate immune response, such as macrophages and dendritic cells, triggering the inflammatory response.

The presence of HMGB1, released in the extracellular matrix following inflammatory stimuli, appears to significantly affect the intestinal barrier function by altering the permeability of the epithelial intestinal cells and leading to an increased entry of microbial antigens. In fact, in vitro and in vivo studies have correlated the presence of HMGB1 secreted by immunostimulated enterocytes, or by other immune cells, and intestinal barrier dysfunctions (10-15). Furthermore, due to the inflammatory cytokines release, HMGB1 is also potentially involved in the colon inflammation, as demonstrated in animal models (16, 17), and in necrotizing forms of colitis (18,19).

The decrease of secreted HMGB1, by anti-HMGB1 molecules, appears to correlate with an improvement both in the damage of the intestinal barrier and in the mucosal inflammation (11, 13, 14, 16, 19, 20).

The presence and amounts of HMGB1 protein in tissue samples obtained from patients has been already used as a diagnostic and prognostic marker of bowel cancer, particularly of colon and rectum cancers as described in U.S. patent application 2006/0188883. However, it is well known that the cancer disease is a very different condition from inflammatory bowel disease. Furthermore, the object of the patent application is applicable only in the use of biological tissues and no reference is provided in connection with the use of fecal material.

In a recent article by Dave et al., (16) results concerning the use of an anti-inflammatory agent such as ethyl pyruvate in a mouse model of chronic colitis to reduce the HMGB1 secretion have been shown. Tests carried out on fecal samples have shown that HMGB1 levels in the stool decrease following the administration of ethyl pyruvate.

However, experiments carried out in the Dave's study on colitis only refer to a mouse model, and it is known that not always the results thus obtained can be automatically extended to humans and their diseases; actually very often the results obtained by using animal models do not coincide at all to the corresponding human disease, both in terms of molecular markers, and in the clinical course of the disease and in the response to specific treatments.

Furthermore, the mouse model used in this study is also employing a genetically modified strain of mice with the gene coding for IL-10 deleted, an anti-inflammatory cytokine, causing colitis in the mice. This is a rather unlike condition compared to the human disease wherein much more complex contributory factors determine the onset of the disease.

In fact, it is well known that genetic and environmental variability characterizing the humans, is absolutely not reproducible in laboratory animal models. In particular, the inflammatory bowel diseases are multifactorial diseases where the genetic and environment variability play an important role in the onset and development of the disease.

In fact, to date, more than thirty susceptibility loci for CD have been identified and less for CU, furthermore not all affected individuals express the same gene variants, as well as, having the gene variant does not necessarily imply developing the disease: i.e. there is a large genetic variability among people having the inflammatory disease, unlike the mouse model where the genetic homogeneity is almost total.

In addition, the environmental pressure, in terms of lifestyle (diet, smoking, stress), as well as the use of drugs or the exposure to harmful environmental agents, differs from person to person and also plays a role in the onset of the disease, the composition of the intestinal flora is different from individual to individual as well. In this regard, it is important to recall that very recent studies conducted by important national and international groups emphasize the key role of commensal bacterial flora in the inflammatory bowel disease, which in fact is altered in affected compared to healthy individuals. Again, the mouse model, in standard conditions, does not suffer at all, or at least much less, the environmental pressure, also the microbiological profile is much less variable among individuals, receiving the same diet.

Role of HMGB1 in Human Intestinal Inflammation

There are very few studies regarding the role of HMGB1 in bowel inflammation in humans: a recent publication indicates ligands of RAGE, including HMGB1 therefore, as potential “biomarkers” of pathological conditions such as arthritis and colitis (21), a second publication identifies HMGB1 as a new antigen of ANCA (Anti-neutrophil cytoplasmic antibody), as observed in the serum of patients with ulcerative colitis (22).

Proteins Used as Markers of Intestinal Inflammation

Biological markers represent a non-invasive method to objectively measure the inflammation and may play a primary or secondary role in the evaluation of some diseases (23), including inflammatory bowel disease (IBD, “Inflammatory Bowel Disease”).

Such markers can be identified as serological or fecal and be used to diagnose a specific process, to classify the disease into different subtypes, to evaluate its activity, evolution and prognosis, to predict the response to a therapeutic treatment or a recurrence (24).

Available serological markers for several inflammatory diseases, including IBDs, are: the erythrocyte sedimentation rate (ESR), C-reactive protein (CRP), the anti-neutrophil cytoplasmic antibodies (ANCA) and the anti-Saccharomyces cerevisiae antibodies (ASCA) (24). However, they show low sensitivity and specificity for intestinal inflammation and poorly correlate with symptoms and indices of disease activity (24).

In contrast, fecal markers show greater specificity for the diagnosis of gastrointestinal diseases, such as IBD, because their levels do not increase in diseases not involving the digestive system (25, 26); furthermore they have the advantage of not necessarily requiring the endoscopic analysis to assess the disease activity (26, 27). Lactoferrin and calprotectin are at the time the most used fecal markers for bowel inflammation (24, 25, 28, 29). In fact, the presence of these proteins in the stool is a reasonably accurate measure of disease activity, recurrence prediction, and identification of high-risk groups among patients with severe colitis and monitoring on the effects of medical therapies.

Given the increasing need to identify methods to detect gastrointestinal inflammation non-invasive, more sensitive and specific, but economic as well, much attention continues to be addressed to the identification of new molecules that comply these characteristics.

### OBJECTIVE AND PRELIMINARY RESULTS

Given the well-known ability of HMGB1 to release signals directed to the recruitment of cell inflammatory repertoire and to activate the immune response due to exogenous or endogenous stimuli, the inventors have proposed to investigate the possible involvement of this protein in the pathogenesis of human inflammatory bowel diseases, more particularly CD and UC.

The CD is characterized by transmural inflammation that can affect any section of the digestive tract, from mouth to anus. Typically there is the involvement of more sections in a discontinuous way. The inflammation involves the entire wall of the affected section and often spreads to nearby mesentery and lymph nodes. Most frequently it involves the terminal ileum and colon.

In UC the inflammatory process is restricted to the colon and affects only the mucosa. The involvement of the rectum is constant and can be accompanied by the involvement of a variable upstream section of the colon.

Currently the prevalence of these diseases in Western countries (Europe and North America) is around 70-150 cases every 100,000 inhabitants for UC and 20-40 cases every 100,000 inhabitants for CD. They are primarily diseases of late adolescence and young-adult age, with a peak onset between 15 and 35 years.

In this context, great importance has been given by the inventors to the discovery of HMGB1 in the stool of pediatric patients with IBD, as it is known that the protein exerts its inflammatory activity when secreted in the extracellular matrix, and feces are precisely what is produced and eliminated from the gut. The obtained data have been then compared with those of a control group.

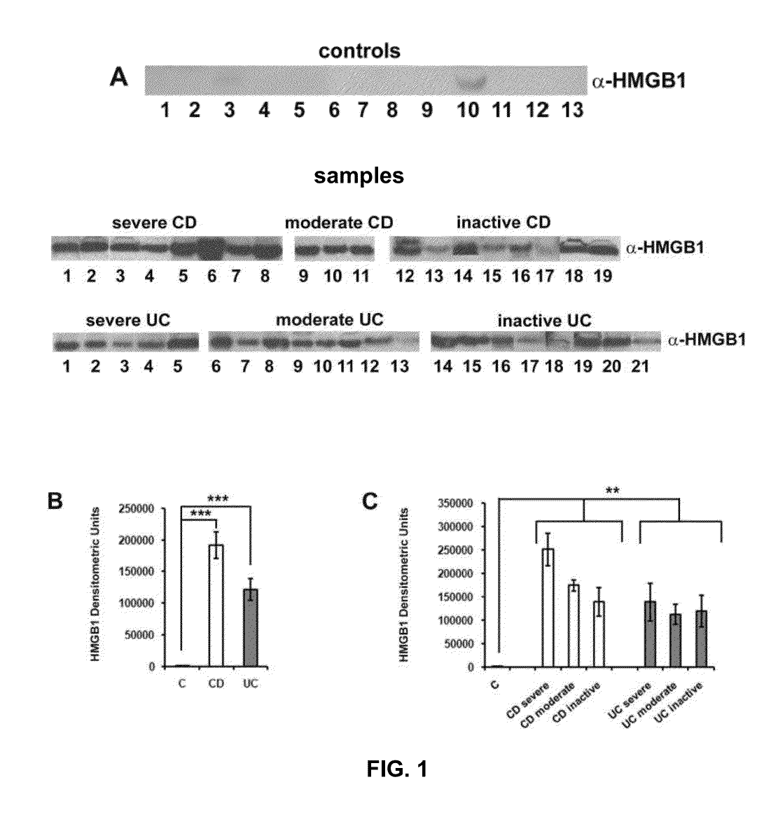

It was surprisingly found that HMGB1 levels observed in the feces of patients with IBD were significantly increased compared to those of healthy controls (FIG. 1). This has allowed to establish that the determination of HMGB1 in the stool of a patient can be used as a marker of intestinal inflammation. In addition, it has become clear that patients with a moderate severity of illness (group with index of disease PCDAI/PUCAI≦25/60), because undergoing treatment, show a reduced presence of HMGB1 compared to those with severe disease. Therefore, this protein, besides being a good marker of inflammation, also seems to be a good indicator of response to therapy (FIG. 1). The methodology developed for this purpose is illustrated below.

Sampling

Fecal samples collected from 40 pediatric patients affected by IBD, respectively, 19 with Crohn's disease (CD) and 21 with ulcerative colitis (UC), plus 13 controls, have been analyzed to assess the presence of HMGB1 by Western blot. The conditions of Western blots have been specifically developed for this purpose for which two specific antibodies for the detection of HMGB1 have been used.

The bands highlighted related to the presence of HMGB1 protein were subjected to densitometric analysis performed by using the ImageQuant software (GE Healthcare Life Sciences, Uppsala, Sweden); thus it was possible to assign a numerical value to range of HMGB1 levels.

The diagnosis of IBD in patients was performed according to endoscopic and histological criteria widely recognized and shared (30). The activity of the CD was measured by the “pediatric Crohn's disease activity index” (PCDA), a measure based on clinical and laboratory parameters (31): the disease is considered inactive if the value is ≦10, mild to moderate if the value is >10-30 and severe if the value is >30. The activity of the UC was ranked according to the “pediatric ulcerative colitis activity index” (PUCAI) (32): the latter is a multi-parametric method recently validated, non-invasive, according to which the disease is considered in remission (score lower than 10), mild disease (score between 10 and 34), moderate (score between 35 and 64) and severe disease (score between 65 and 85).

The endoscopic score was determined using the SES-CD (33) and Matts' score (34) for ulcerative colitis. To calculate the SES-CD, the bowel was divided into five segments (ileum, left colon, transverse colon, right colon, rectum), and to the degree of disease activity in each segment was assigned a value ranging from 0 to 12 (total value range: 0-60). To calculate the Matts' score the intestine was divided into six segments (blind, ascending colon, transverse colon, descending colon, sigmoid colon, rectum), and in each segment a value that ranges from 1 to 4 (total value range: 6-24) was assigned to the degree of disease activity.

Based on these indices in the enrolled patients in the study the disease has been found severe in 13 IBD (8 CD and 5 UC), mild to moderate in 11 patients (3 CD and 8 UC) and inactive in 16 (8 CD and 8 UC) (Table 1).

Table 1 lists the patients, divided according to disease type and severity, enrolled in the clinical trial.

TABLE 1
Demographic characteristics and indexes
of disease activity of IBD in the studied patient
population. PCDAI: “Pediatric Crohn's Disease Activity
Index” PUCAI: “Pediatric Ulcerative Colitis Activity
Index”

| Subject | Sex | Age (years) | PCDAI |
|---|---|---|---|
| Severe disease | | | |
| MC1 | F | 10 | 57 |
| MC2 | M | 17 | 55 |
| MC3 | M | 10 | 35 |
| MC4 | M | 12 | 35 |
| MC5 | F | 12 | 35 |
| MC6 | M | 15 | 30 |
| MC7 | F | 17 | 35 |
| MC8 | F | 17 | 25 |
| Moderate disease | | | |
| MC9 | M | 15 | 22 |
| MC10 | M | 9 | 17 |
| MC11 | M | 13 | 15 |
| Inactive disease | | | |
| MC12 | F | 13 | 10 |
| MC13 | F | 16 | 10 |
| MC14 | M | 10 | 10 |
| MC15 | M | 14 | 10 |
| MC16 | M | 11 | 10 |
| MC17 | M | 18 | 5 |
| MC18 | M | 16 | 5 |
| MC19 | M | 12 | 5 |
| Subject | Sex | Age (years) | PUCAI |
| Severe disease | | | |
| CU1 | F | 14 | 80 |
| CU2 | F | 10 | 75 |
| CU3 | M | 12 | 75 |
| CU4 | F | 7 | 65 |
| CU5 | M | 11 | 65 |
| Moderate disease | | | |
| CU6 | F | 14 | 60 |
| CU7 | M | 11 | 60 |
| CU8 | M | 15 | 50 |
| CU9 | M | 11 | 40 |
| CU10 | F | 14 | 40 |
| CU11 | F | 17 | 25 |
| CU12 | M | 13 | 15 |
| CU13 | M | 11 | 15 |
| Inactive disease | | | |
| CU14 | F | 18 | 10 |
| CU15 | F | 18 | 5 |
| CU16 | F | 11 | 5 |
| CU17 | M | 12 | 0 |
| CU18 | M | 10 | 0 |
| CU19 | F | 10 | 0 |
| CU20 | F | 7 | 0 |
| CU21 | M | 14 | 0 |

Preparation of Fecal Sample

Fecal samples were obtained from pediatric patients affected by IBD (Table 1), with varying degrees of severity of illness, and from healthy controls, recruited at the Department of Pediatrics, Pediatric Gastroenterology and Hepatology Unit, University of Rome “La Sapienza”, directed by Professor Salvatore Cucchiara.

The samples, collected in sterile containers for feces, were stored at a temperature between −20° C. and −80° C. until the molecular analysis.

Weighing And Suspension of Feces Sample in Buffer Solution

Each sample (equivalent to the contents of the spoon inside a standard container for feces) was removed with a sterile tip from the container, put in a 1.5 ml eppendorf tube and weighed using a digital scale. The sample was resuspended in extraction buffer (phosphate buffered saline solution PBS pH 7.2) marketed by the company ScheBo Biotech containing detergent and sodium azide, to obtain a final concentration of 500 mg/ml.

Homogenization And Extraction of the Stool

The sample was vortexed for one minute at room temperature (RT) and then placed in an orbital shaker at room temperature for about one hour. Following centrifugation for 5 minutes at 10000 rpm at 4° C., the supernatant, defined extracted fecal, was collected and the protein concentration was measured by Bradford assay (Biolabs). The sample obtained can be immediately analyzed by Western blot assay or stored at −80° C. and subsequently analyzed.

Analysis of Fecal Extracts by Western Blot

To 20 μg of fecal protein extract an equal volume of 2× Sample Buffer (100 mM Tris-Cl pH 6.8, 10% beta-mercaptoethanol, 4% SDS, 20% glycerol, 0.2% bromophenol blue) was added; the sample was then boiled for 5 minutes and centrifuged briefly before proceeding to the analysis of the extracts by Western blot (WB). The fecal protein extract was separated using 12% SDS-polyacrylamide gel and then transferred onto a PVDF filter (Amersham), by electrotransfer, for 1 hour at 70 volts. Non-specific sites on the filter were blocked by incubation for 1 hour at room temperature with Blocking Buffer (0.02M Tris-Cl pH 7.6, 0.137M NaCl, 5% fat-free milk powder), then the filter was incubated for 16 hours at 4° C. with anti-HMGB1 polyclonal antibody (Cat. No. H9593, Sigma), diluted 1:1000 in Antibody Buffer (0.02M Tris-Cl pH 7.6, 0.137M NaCl, 3% fat-free milk powder) or with anti-HMGB1 monoclonal antibody (Cat. No. MAB 1690, R&D Systems, Minneapolis, USA) diluted 1:500 in Antibody Buffer. Three washes of five minutes each in TBS-T 0.1% Tween (0.02M Tris-Cl pH 7.6, 0.137M NaCl, 0.1% Tween) were then carried out, and subsequently, the filter was incubated for 1 hour at room temperature with an anti-rabbit secondary antibody when using anti-HMGB1 from Sigma, an anti-mouse secondary antibody was used in case of incubation with anti-HMGB1 antibody R&D System, both conjugated to peroxidase (Santacruz), diluted 1:4000 in Antibody Buffer.

The polyclonal anti-HMGB1 antibody (Cat. No. H9593, Sigma) is produced in rabbit using as immunogen a synthetic peptide corresponding to amino acid 165-180 of human HMGB1, whereas the monoclonal anti-HMGB1 antibody (Cat. No. MAB 1690, R&D System, Minneapolis, USA) corresponds to clone 115603 of the hybridoma resulting from the fusion of a mouse myeloma with B cells obtained from a mouse immunized with purified E. coli derived recombinant human HMGB1 protein.

Three additional washes of 5 minutes were carried out in TBS-T+0.1% Tween and then it was proceeded to the detection of the chemiluminescent signal using ECLplus (Amersham) and autoradiographic films (Kodak).

FIG. 1 shows the HMGB1 protein in stool samples, detected by Western blot analysis using anti-HMGB1 monoclonal antibody of R&D System. In particular, panel A shows the result of Western blot, panel B shows a graph of the densitometric values of the highlighted bands by Western blot in patients, panel C shows a graph of the densitometric values of the highlighted bands by Western blot in patients divided in groups according to the severity of the disease.

The densitometric analysis of the highlighted bands by Western blot has allowed to obtain a numerical value related to the level of HMGB1 present in the fecal sample. In particular, it has been found that in healthy individuals such numeric value ranges from 1000 to 3000, expressed as Arbitrary Densitometric Units (ADU), mean value 1200 ADU; subjects with CD show a numeric value ranging from 20000 to 380000 ADU, average value 190000 ADU; while all patients affected by UC show a numeric value related to the level of HMGB1 present in analyzed fecal samples ranging from 6000 to 280000 ADU, with average value equal to 120000 ADU (FIG. 1-B). In the figure the asterisks refer to statistical significance evaluated through the Mann-Whitney statistical test: where *p<0.05, **p<0.01, ***p<0.001.

The analysis shows that the HMGB1 protein expression is significantly increased in the feces of IBD patients compared to controls, where is not detectable (p<0.001) (FIG. 1). This result indicates that the presence of HMGB1 protein detected in the feces is a marker of inflammation in the human gut. The presence of HMGB1 protein has been also detected in the feces of 16 patients with disease defined inactive on the basis of the PCDAI and PUCAI indices; however, according to the assessment of the endoscopic score, these patients had some extent of intestinal inflammation, in agreement with the detection of HMGB1 in their feces.

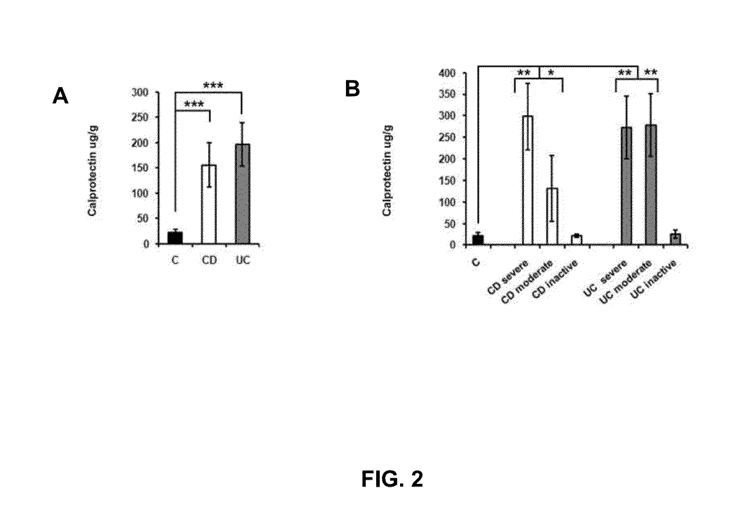

In particular, in patients with active CD and UC, the medians of the SES-CD and of the Matts' scores were respectively 23.0 (range values: 14-34) and 18.0 (range of values: 8-24), and in patients with inactive CD and UC the medians of the SES-CD and of the Matts' scores were respectively 7.5 (range of values: 0-15) and 11.5 (range of values: 6-18). These indices show that even in the so defined inactive patients according to the PCDAI and PUCAI indices there is a pitch of intestinal inflammation, and that HMGB1 may also provide an indication of the inactive state of the disease and therefore regarded as very sensitive marker for such inactive inflammatory conditions. The HMGB1 protein levels detected in the feces were compared with those of fecal calprotectin, which is currently considered a biomarker of choice and reliable for the diagnosis of intestinal inflammation by ELISA (29, 35). The results of this analysis are shown in FIG. 2, where the asterisks indicate statistical significance according to the statistical test of Mann-Whitney, where *p<0.05, *p<0.01, ***p<0.001. Both proteins resulted to be strongly increased in the feces of patients (p<0.001) compared with healthy controls (FIG. 1-B, FIG. 2-A). However, the inactive CD and UC groups showed a low level of calprotectin, but a significant increase in fecal levels of HMGB1 compared to controls (p<0.01 in CD and UC) (FIG. 1-C, FIG. 2-B). In summary, the comparison has shown a significant correlation between the levels of the two proteins in fecal samples in all patients with a diagnosed active inflammatory disease, both CD and UC (r: 0.77 in CD, r: 0.70 in UC, p<0.01) with r=rank correlation coefficient according to Spearman's test. Such correlation disappeared when only patients with inactive inflammatory disease were considered (r: 0.22 in CD, r: 0.18 in UC, not symptomatic). Indeed, HMGB1 is significantly elevated in all 16 patients with so defined inactive disease according to the indices of PCDAI and PUCAI, despite they still show a degree of inflammation in accordance with the endoscopic score, while calprotectin was elevated in only two of them. This would indicate that HMGB1 is a very sensitive marker of persistent intestinal inflammation in patients with clinically quiescent disease, as revealed by classical indices of disease activity. The latter, however, being a mixture of the clinical and laboratory features, are not always correlated with intestinal inflammation detected by endoscopy.

It is therefore conceivable the use of the HMGB1 protein as a potential molecular prognostic parameter for recurrence in patients with disease in apparent remission. In FIG. 1, patients with IBD show a significant increase of HMGB1 in the feces compared to healthy controls. In addition, there is a direct correlation between HMGB1 levels and severity of the disease. In conclusion, HMGB1, beside being a good marker of inflammation, seems also to provide a good indicator of the severity of the disease and, therefore, could be used as a marker of response to therapy.

From what has been described here is evident the importance of the present invention: the use of HMGB1 as a biological marker and the method to detect its presence in stool samples is a significant step forward in order to diagnose in a safe and non-invasive manner the presence and the level of a human intestinal inflammation, avoiding the often repeated imaging studies that are highly traumatic for most patients.

Furthermore, the protein expression levels can be used as prognostic marker of recurrence of disease and as a marker of response to therapy.

It is worth noting that for the analysis of fecal extracts not only the Western blot assay can be used. The inventors have indeed turned their attention to the development of an analysis protocol to detect the presence of HMGB1 in feces by means of ELISA, using the same antibodies used for Western blot assay which has given more than decent results in terms of specificity and sensitivity to the target protein. Therefore, it is proceeding with the construction of an ELISA kit using the two antibodies already used in the detection of protein by WB in stool samples. The choice to provide an ELISA protocol, in addition to the Western blot, is dictated by the fact that this technique is simple and moreover allows to better quantify the reaction, in fact, the colour intensity of the ELISA plate is proportional to the number of antigen-antibody complexes (primary) and thus to the concentration of antigen (capable of binding the primary antibody) in the analyzed sample.

### BIBLIOGRAPHY

1. Wang et al. Am J Respir Crit. Care Med. 164:1768-73, (2001)
2. Fink et al. J Intern Med. 261:349-62, (2007)
3. Hirschfeld at al. J. Immunol. 165:618-22, (2000)
4. Andersson et al. J Exp Med. 192: 565-570, (2000)
5. Scaffidi et al. Nature. 418:191-5, (2002)
6. Bianchi et al. J Leukoc Biol. (2009)
7. Taniguchi et al. Arthritis Rheum. 48:971-81, (2003)
8. Jiang et al. Ann Rheum Dis. 67:727-8, (2008)
9. Ulfgren et al. Arthritis Rheum. 50:1586-94, (2004)
10. Luan Z G et al. Pancreas 39:216-23 (2010)
11. Fink M. Crit. Care Med (2009)
12. Yang R et al. Am J Physiol Regul Integr Comp Physiol 297: R362-9 (2009)
13. Wu R et al. Crit. Care Med 37:2483-5 (2009)
14. Liu S et al. Am J Physiol Cell Physiol 290: C990-9 (2006)
15 Sappington et al. Gastroenterology. 123:790-802, (2002)
16. Dave S H et al. J Leukoc Biol 86:633-43 (2009)
17. Maeda S et al. Biochem Biophys Res Commun 360:394-400 (2007)
18. Dai S et al. J Biol Chem 285:4995-5002. (2009)
19. Zamora R et al. Am J Physiol Gastrointest Liver Physiol 289:G643-52 (2005)
20. Yang R et al. Mol Med 12:105:14 (2006)
21. Clynes R et al. Curr Mol Med 7:743-51 (2007)
22. Ito I et al. J Biol Chem 282:16336-44 (2007)
23. Desai D et al. Aliment Pharmacol Ther 25:247-255 (2007)
24. Gisbert J P et al. Gastroenterol Hepatol 30:117-129 (2007)
25. Angriman I et al. Clin Chim Acta 381:63-68 (2007)
26. Tibble J A, Bjarnason I. Fecal Drugs Today (Barc) 37:85-96 (2001)
27. Vermeire S et al. Gut 55:426-431 (2006)
28. Sutherland A D et al. Dis Colon Rectum 51:1283-1291 (2008)
29. Gisbert J P, McNicholl A G. Dig Liver Dis 41:56-66 (2009)
30. Bousvaros A, Antonioli D A, Colletti R B et al. Pediatr Gastroenterol Nutr 44:653-74 (2007)
31. Hyams J S, Mandel F, Ferry G D, et al. J Pediatr Gastroenterol Nutr 12:439-47 (1991)
32. Turner D, Otley A R, Mack D, et al. Gastroenterology 133:423-32. (2007)
33. Daperno M, D'Haens G, Van Assche G, et al. Gastrointest Endosc 60:505-12 (2004)
34. Osada T, Ohkusa T, Yokoyama T, et al. Inflamm Bowel Dis 16:192-197 (2010)
35. Diamanti A, Panetta F, Basso M S et al. Inflamm Bowel Dis 16:1926-30 (2010)

1-12. (canceled)

13. A non-invasive method for detecting, diagnosing and prognosing bowel inflammatory conditions in a human patient, characterized in that it detects the level of HMGB1 protein in fecal samples of the same patient.

14. The non-invasive method according to claim 13, characterized in that it uses specific anti-HMGB1 antibodies to detect the level of HMGB1 in fecal samples.

15. The non-invasive method according to claim 13, wherein the specific anti-HMGB1 antibodies are polyclonal anti-HMGB1 antibodies and monoclonal anti-HMGB1 antibodies.

16. The non-invasive method according to claim 15, characterized in that said polyclonal anti-HMGB1 antibody is produced in rabbit using as immunogen a synthetic peptide corresponding to amino acid 165-180 of human HMGB1.

17. The non-invasive method according to claim 15, characterized in that said monoclonal anti-HMGB1 antibody corresponds to clone 115603 of the hybridoma resulting from the fusion of a mouse myeloma with B cells obtained from a mouse immunized with purified E. coli derived recombinant human HMGB1 protein.

18. The non-invasive method according to claim 13 wherein the level of HMGB1 in the fecal sample is revealed by the specific antigen-antibody reaction in Western blot analysis and/or ELISA assay.

19. The non-invasive method according to claim 13, comprising the following steps:
weighting of the fecal sample and suspension in PBS extraction buffer;
homogenization of the sample and extraction after centrifugation of the supernatant fecal extract;
evaluation of the protein concentration by Bradford assay;
detection of HMGB1 protein level in the fecal extract by Western blot or ELISA assay.

20. The non-invasive method according to claim 13, wherein said bowel inflammatory conditions are selected from the group consisting of chronic inflammatory bowel diseases (IBD), specifically Crohn's disease (CD) and colitis ulcerous (CU).

21. The non-invasive method according to claim 13, wherein the human patient is a pediatric patient suffering from IBD.

22. The non-invasive method according to claim 13, wherein the decrease of the level of HMGB1 protein in the fecal sample is used as response marker to a given treatment for IBD.

23. Anti-HMGB1 antibodies for detecting, diagnosing and prognosing bowel inflammatory conditions in a human patient, wherein the anti-HMGB1 antibodies are capable of detecting the level of HMGB1 protein in fecal samples.

24. The anti-HMGB1 antibodies according to claim 23, wherein the anti-HMGB1 antibodies are polyclonal anti-HMGB1 antibodies and monoclonal anti-HMGB1 antibodies.

25. The anti-HMGB1 antibodies according to claim 24, characterized in that said polyclonal anti-HMGB1 antibody is produced in rabbit using as immunogen a synthetic peptide corresponding to amino acid 165-180 of human HMGB1.

26. The anti-HMGB1 antibodies according to claim 24, characterized in that said monoclonal anti-HMGB1 antibody corresponds to clone 115603 of the hybridoma resulting from the fusion of a mouse myeloma with B cells obtained from a mouse immunized with purified E. coli derived recombinant human HMGB1 protein.

27. The anti-HMGB1 antibodies according to claim 23, wherein the level of HMGB1 in the fecal sample is revealed by the specific antigen-antibody reaction in Western blot analysis and/or ELISA assay.

28. The anti-HMGB1 antibodies for detecting, diagnosing and prognosing bowel inflammatory conditions in a human patient, according to claim 23, in a method comprising the following steps:
weighting of the fecal sample and suspension in PBS extraction buffer;
homogenization of the sample and extraction after centrifugation of the supernatant fecal extract;
evaluation of the protein concentration by Bradford assay;
detection of HMGB1 protein level in the fecal extract by Western blot or ELISA assay.

29. The anti-HMGB1 antibodies for detecting, diagnosing and prognosing bowel inflammatory conditions in a human patient, according to claim 23, wherein said bowel inflammatory conditions are selected from the group consisting of chronic inflammatory bowel diseases (IBD), specifically Crohn's disease (CD) and colitis ulcerous (CU).

30. The anti-HMGB1 antibodies for detecting, diagnosing and prognosing bowel inflammatory conditions in a human patient, according to claim 23, wherein the human patient is a pediatric patient suffering from IBD.

31. The anti-HMGB1 antibodies for detecting, diagnosing and prognosing bowel inflammatory conditions in a human patient, according to claim 23, wherein the decrease of the level of HMGB1 protein in the fecal sample is used as response marker to a given treatment for IBD.
